(19) United States
(12) Patent Application Publication
(10) Pub. No.: US 2011/0257528 A1
(43) Pub. Date: Oct. 20, 2011

(54) DIAGNOSTIC SONOGRAPHIC SYSTEM FOR ECHOGRAPHIC EXPLORATION OF HUMAN ORGANS AND METHOD OF SIMULATING SURGERY USING SAME

(76) Inventor: JUNG-TUNG LIU, Taichung City (TW)

(21) Appl. No.: 12/761,235

(22) Filed: Apr. 15, 2010

(51) Int. Cl. A61B 8/14

(52) U.S. Cl. 600/443

(57) ABSTRACT

A diagnostic sonographic system for echographic exploration of human organs includes a transducer assembly comprising a first transducer module including a first sound wave generation and transmission member and a first echo receiver, a second transducer module including a second sound wave generation and transmission member and a second echo receiver, and a through hole between the first and second transducer modules; and an image processing assembly for receiving echoes from the first and second echo receivers and processing and transforming same into a digital image. A method of simulating surgery using same is also provided.

### BACKGROUND OF THE INVENTION

1. Field of Invention

The invention relates to sonography and more particularly to a diagnostic sonographic system for echographic exploration of human organs and method of simulating surgery using same.

2. Description of Related Art

In physics, the term “ultrasound” applies to all acoustic energy (longitudinal, mechanical wave) with a frequency above the audible range of human hearing. The audible range of sound is 20 hertz-20 kilohertz. Ultrasound has a frequency greater than 20 kilohertz.

Diagnostic sonography (i.e., ultrasonography) is an ultrasound-based diagnostic imaging technique used to visualize subcutaneous body structures including tendons, muscles, joints, vessels and internal organs for possible pathology or lesions. There is a plethora of diagnostic and therapeutic applications practiced in medicine. It is possible to perform both diagnosis and therapeutic procedures, using ultrasound to guide interventional procedures (e.g., biopsies or drainage of fluid collections). Sonographers are medical professionals who perform scans for diagnostic purposes. Sonographers typically use a hand-held probe (called a transducer) that is placed directly on and moved over the patient.

U.S. Pat. No. 6,837,855 discloses “Use of ultrasonic transducer for echographic exploration of human or animal body tissues or organs in particular of the eyeball posterior segment.” Further, U.S. Pat. No. 5,776,068 discloses “Ultrasonic scanning of the eye using a stationary transducer.”

However, as far as the present inventor is aware, there are no documents disclosing a diagnostic sonographic system for echographic exploration of human organs (e.g., brain, head, breast, livers, etc.) and a method of simulating surgery using same for simulation by a, for example, medical professional or intern. Thus, the need for improvement still exists.

### SUMMARY OF THE INVENTION

It is therefore one object of the invention to provide a diagnostic sonographic system for echographic exploration of human organs comprising a transducer assembly comprising a first transducer module including a first sound wave generation and transmission member and a first echo receiver, a second transducer module including a second sound wave generation and transmission member and a second echo receiver, and a through hole between the first and second transducer modules; and an image processing assembly for receiving echoes from the first and second echo receivers and processing and transforming same into a digital image.

It is another object of the invention to provide a method of simulating surgery using a diagnostic sonographic system for echographic exploration of human organ, the diagnostic sonographic system including a transducer assembly having a first transducer module with a first sound wave generation and transmission member and a first echo receiver, a second transducer module with a second sound wave generation and transmission member and a second echo receiver, and a through hole between the first and second transducer modules; an image processing assembly for receiving echoes from the first and second echo receivers and processing and transforming same into a digital image, the image processing assembly having a first amplification element, a first filter, a second amplification element, a second filter, and an imaging unit including a comparison element and an imaging element; and a computer monitor, the method comprising the steps of placing the transducer assembly on a simulated organ and moving over it; activating the transducer assembly, the image processing assembly, and the computer monitor; causing each of the first and second sound wave generation and transmission members to generate and transmit a sound wave toward the simulated organ wherein the sound waves travel into the simulated organ and come into focus at a predetermined depth; returning a portion of reflections from the simulated organ to the first and second echo receivers; causing the first and second echo receivers to send the reflections to the first and second amplification element for amplification respectively; sending the amplified reflections to the first and second filters for transforming into analog video signals respectively; causing the comparison element to compare the analog video signals from the first filter with that from the second filter wherein if a predetermined portion of the analog video signals from the first filter is the same as that from the second filter then resolution of the predetermined portion of either analog video signals is increased for enhancement; causing the imaging element to process the enhanced predetermined portion of either analog video signals and transform same into a digital image; and sending the digital image to the computer monitor for display such that a medical professional is allowed to insert a tool through the through hole into a target portion of the simulated organ for removal while looking at the computer monitor.

The above and other objects, features and advantages of the invention will become apparent from the following detailed description taken with the accompanying drawings.

### BRIEF DESCRIPTION OF THE DRAWINGS

FIG. 1 is a top view of a transducer employed by a diagnostic sonographic system for echographic exploration of human organs according to the invention;

FIG. 2 is a front view of the transducer with a sound wave being produced by each of two transducer modules and echoes being received from a simulated organ;

FIG. 3 is a block diagram of a image processing assembly according to the invention; and

FIG. 4 is an environmental view of the diagnostic sonographic system being used by an intern for simulating surgery according to the invention.

### DETAILED DESCRIPTION OF THE INVENTION

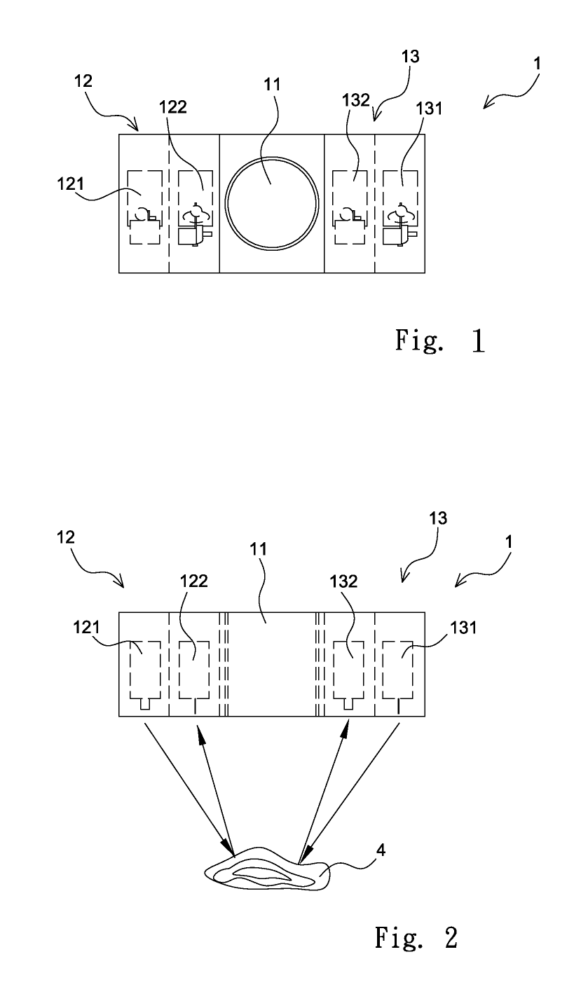

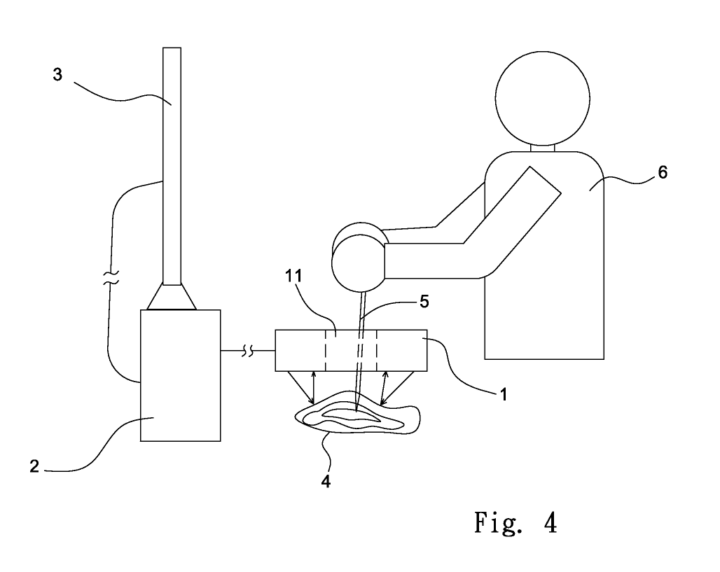

Referring to FIGS. 1 to 4, a diagnostic sonographic system for echographic exploration of human organs in accordance with the invention comprises the following components as discussed in detail below.

A transducer assembly (also called probe) 1 is a rectangular body and comprises a central through hole 11, a first transducer module 12 to the left of the through hole 11, the first transducer module 12 including a first sound wave generation and transmission member 121 and a first echo receiver 122, and a second transducer module 13 to the right of the through hole 11, the second transducer module 13 including a second sound wave generation and transmission member 131 and a second echo receiver 132.

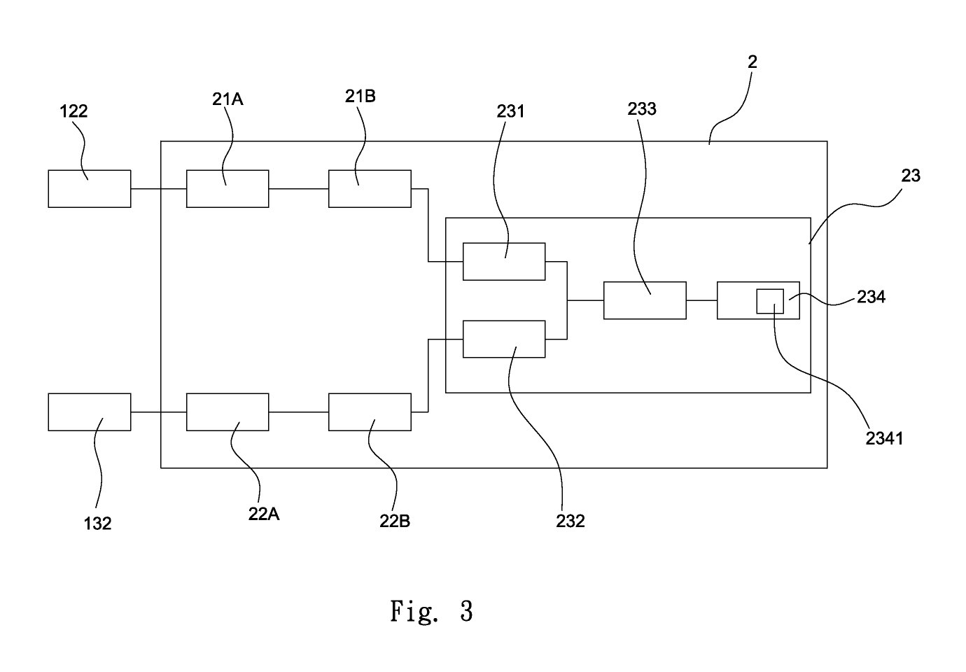

An image processing assembly 2 functions to receive echoes from the first and second echo receivers 122, 132, and process and transform same into a digital image as detailed below. The image processing assembly 2 comprises a first amplification element 21A, a first filter 21B, a second amplification element 22A, a second filter 22B, and an imaging unit 23 including a first memory 231, a second memory 232, a comparison element 233, and an imaging element 234 having a plurality of digital scan converters (DSCs) 2341 (only one being shown in FIG. 3).

In the shown embodiment, the transducer assembly 1 and the image processing assembly 2 are separate. Alternatively, the transducer assembly 1 and the image processing assembly 2 are formed into a unit in another embodiment.

A method of simulating surgery using the diagnostic sonographic system in accordance with the invention comprises the following steps:

For example, a medical intern 6, may place the transducer assembly 1 directly on a simulated organ 4 and move over it. Next, activate the transducer assembly 1, the image processing assembly 2, and a computer monitor 3. The first and second sound wave generation and transmission members 121, 131 each generates and transmits a sound wave toward the simulated organ 4. The sound wave has a lower frequency (e.g., about 2 MHz). The sound waves travel into the simulated organ 4 and come into focus at a desired depth. The sound wave reflects and some of the reflections from the simulated organ 4 return to the first and second echo receivers 122, 132. Next, the first and second echo receivers 122, 132 send the reflections to the first and second amplification element 21A, 22A for amplification respectively. Next, the amplified reflections are sent to the first and second filters 21B and 22B respectively. Next, the filtered, amplified reflections (i.e., analog video signals) are sent to the first and second memories 231, 232 for storage respectively. Next, the comparison element 233 compares the filtered, amplified reflection from the first memory 231 with that from the second memory 232. If a predetermined substantial portion of the filtered, amplified reflection from the first memory 231 is the same as that from the second memory 232 it is concluded that a target portion of the simulated organ 4 (i.e., organ being adversely affected by disease) is found. Next, resolution of the predetermined substantial portion of either filtered, amplified reflection is increased for enhancement. Next, the enhanced predetermined substantial portion of either filtered, amplified reflection is processed and transformed into a digital image by the DSCs 2341. Finally, the digital image is sent to the computer monitor 3 for display.

The medical intern 6 may clearly see the image of the target portion of the simulated organ 4 being adversely affected by disease on the computer monitor 3. Thus, the medical intern 6 may hold a tool (e.g., a pair of tweezers) 5 and insert same through the through hole 11 into a target portion of the simulated organ 4. It is noted that the bore of the through hole 11 is sufficiently greater than that of the pair of tweezers 5 so that the pair of tweezers 5 may freely insert into the through hole 11 and clear same. Next, the medical intern 6 may remove the target portion of the simulated organ 4 by means of the pair of tweezers 5.

In brief, the invention has the following advantages: Two sets of transducer module are employed so that the target portion of the simulated organ can be precisely positioned for simulation purposes. Also, a medical professional or intern may clearly see the target portion of the simulated organ on a computer monitor for removal. Sound wave of lower frequency with strong penetration capability is employed. It has simplified components. It is highly reliable. Its image is high in resolution.

While the invention herein disclosed has been described by means of specific embodiments, numerous modifications and variations could be made thereto by those skilled in the art without departing from the scope and spirit of the invention set forth in the claims.

What is claimed is:

1. A diagnostic sonographic system for echographic exploration of human organs comprising:
a transducer assembly comprising a first transducer module including a first sound wave generation and transmission member and a first echo receiver, a second transducer module including a second sound wave generation and transmission member and a second echo receiver, and a through hole between the first and second transducer modules; and
an image processing assembly for receiving echoes from the first and second echo receivers and processing and transforming same into a digital image.

2. The diagnostic sonographic system of claim 1, wherein the image processing assembly comprises a first amplification element, a first filter, a second amplification element, a second filter, and an imaging unit including a comparison element and an imaging element.

3. The diagnostic sonographic system of claim 2, wherein the imaging element comprises a plurality of digital scan converters (DSCs).

4. The diagnostic sonographic system of claim 1, wherein the transducer assembly and the image processing assembly are separate.

5. The diagnostic sonographic system of claim 1, wherein the transducer assembly and the image processing assembly are formed into a unit.

6. The diagnostic sonographic system of claim 1, wherein a diameter of the through hole is sufficiently greater to allow a tool to access.

7. The diagnostic sonographic system of claim 6, wherein the tool is a pair of tweezers.

8. A method of simulating surgery using a diagnostic sonographic system for echographic exploration of human organ, the diagnostic sonographic system including a transducer assembly having a first transducer module with a first sound wave generation and transmission member and a first echo receiver, a second transducer module with a second sound wave generation and transmission member and a second echo receiver, and a through hole between the first and second transducer modules; an image processing assembly for receiving echoes from the first and second echo receivers and processing and transforming same into a digital image, the image processing assembly having a first amplification element, a first filter, a second amplification element, a second filter, and an imaging unit including a comparison element and an imaging element; and a computer monitor, the method comprising the steps of:
(a) placing the transducer assembly on a simulated organ and moving over it;
(b) activating the transducer assembly, the image processing assembly, and the computer monitor;
(c) causing each of the first and second sound wave generation and transmission members to generate and transmit a sound wave toward the simulated organ wherein the sound waves travel into the simulated organ and come into focus at a predetermined depth;
(d) returning a portion of reflections from the simulated organ to the first and second echo receivers;
(e) causing the first and second echo receivers to send the reflections to the first and second amplification element for amplification respectively;
(f) sending the amplified reflections to the first and second filters for transforming into analog video signals respectively;
(g) causing the comparison element to compare the analog video signals from the first filter with that from the second filter wherein if a predetermined portion of the analog video signals from the first filter is the same as that from the second filter then resolution of the predetermined portion of either analog video signals is increased for enhancement;
(h) causing the imaging element to process the enhanced predetermined portion of either analog video signals and transform same into a digital image; and
(i) sending the digital image to the computer monitor for display such that a medical professional is allowed to insert a tool through the through hole into a target portion of the simulated organ for removal while looking at the computer monitor.

9. The method of claim 8, wherein the imaging element comprises a plurality of digital scan converters (DSCs).
